(19) United States
(12) Patent Application Publication
(10) Pub. No.: US 2002/0042557 A1
(43) Pub. Date: Apr. 11, 2002

(54) Diagnostic / patient demonstration aid

(76) Inventors: William George Bensen, Hamilton (CA); Wynn Grayann Bensen, Hamilton (CA)

Correspondence Address: H. Roger Hart, Bereskin & Parr, 40 King Street West, Box 401, Toronto, ON, M5H 3Y2, CA

(21) Appl. No.: 09/891,329

(22) Filed: Jun. 27, 2001

Related U.S. Application Data
(60) Provisional application No. 60/214,469, filed on Jun. 27, 2000.

(51) Int. Cl. G09B 23/28; A61B 5/00; G09F 1/00

(52) U.S. Cl. 600/300; 434/262; 40/446

(57) ABSTRACT

A diagnostic tool to assist patients in understanding the nature of medical conditions such as arthritis, includes a graphic display having a central scale with end points showing “REMISSION” and “WORST”. In addition, the tool comprises scales placed on either side and substantially equidistant from the central scale. One of the side scales is a patient assessment scale while the other scale is a clinical assessment scale. The side scales are arranged with end points adjacent the end points of the central scale. Lines may be drawn on the scale joining a patient's self assessment point with a clinical assessment done at the same time to demonstrate to the patient the patient's present condition, the therapeutic potential and the therapeutic opportunity.

### CROSS-REFERENCE TO RELATED APPLICATIONS

This application claims benefit from U.S. Provisional Application Ser. No. 60/214,469 filed Jun. 27, 2000 which is incorporated herein by reference in its entirety.

### FIELD OF THE INVENTION

This invention relates to diagnostic methods for use in the treatment of human beings. In many diseases and in particular in the field of rheumatology there is a lack of a tool that can be used when a patient is present in a clinic that will give a sense of where the patient is with their disease, what can be done for the patient and what steps the patient might take. Heretofore there have been developed extensive questionnaires. These questionnaires function as a form of status report. These status reports do not easily permit comparison nor do they encourage change on the part of either the patient or the physician. Such a status report, although helpful does not provide a perspective to the patient as to where they stand in the context of how much worse or better the patient's condition might be.

It has been observed that in many cases when a patient responds to a status inquiry by advising that they are ok or better, the medical professionals involved seldom make any changes. For various reasons it is usually only when a patient states that they are worse off that the physician will take steps to change the status quo. In this respect the traditional patient response usually determines the medical response thereto. Because of this lack of relation of what the therapeutic opportunity or short fall is and the full range of therapeutic potential between best and worst, often steps are not taken when such steps could be taken with advantageous effect for the patient. Accordingly, there is need for a diagnostic tool which assists both the patient and their health care provider to better understand the treatment potential which may be available to the particular patient.

The diagnostic tool of this invention is applicable to many diseases of humans including rheumatoid arthritis, osteo arthritis osteoporosis as well as hypertension, asthma and a number of different diseases. While the basic concepts for each disease remain similar, the calibrations as more fully explained below are different, and possibly specific to each disease condition.

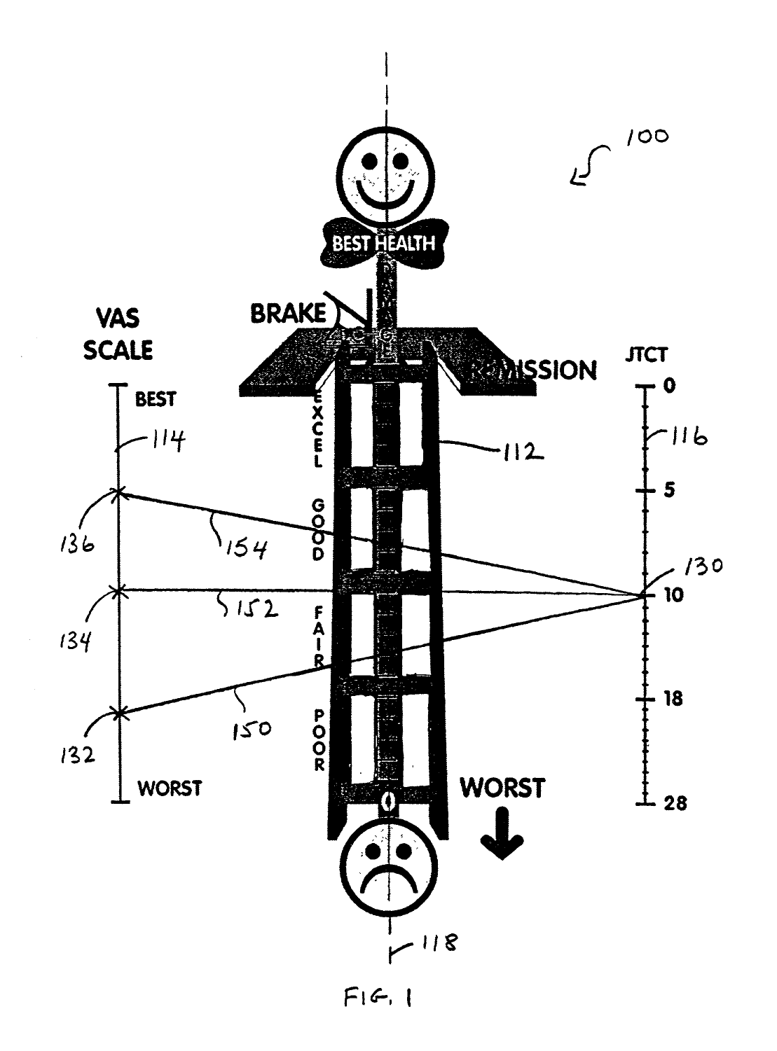

The basic concept of the diagnostic tool is a three part scale. A scale for use in association with treatment of rheumatoid arthritis is illustrated in FIG. 1. This scale may be used for both rheumatoid and osteo arthritis.

The diagnostic tool indicated generally at 100 comprises three scales 112, 114 and 116. The first scale 112 is generally centrally located with scales 114 and 116 on opposite sides of the central scale 112. The central scale in the case of arthritis contains a range from worst, to remission. There are a series of gradations on a linear scale between the end points “WORST” and “REMISSION”. The scale 112 has a general axis 118.

The central scale 112 is presented to the patient in the form of a ladder extending between the end points “WORST” and “REMISSION”. At the upper end of the ladder there is a depiction of a platform which is labelled “REMISSION”. The platform also may conveniently have a symbol for a “BRAKE”. At the upper end of the scale 112 there is also the word “DAMAGE” together with the words “BEST HEALTH” and a happy face. At the lower end of the scale there is a glum face. This particular presentation of the central scale, largely artistic in nature, provides a presentation of the patient's condition to the patient in a form which is readily understandable to the patient.

Any patient with arthritic disease will have some damage. In order to prevent further damage to the patient's body the progression of the disease must be stopped or braked. Thus, what is presented to the patient is that their best case scenario is to reach the “REMISSION” platform and put the “BRAKE” on the disease to inhibit further progress of the disease.

A second scale 114 is arranged with an axis that is generally parallel to the first scale 112. The second scale 114 comprises a line having two end points. One end point is identified “WORST”, while the second end point is identified “BEST”. The end point “WORST” on scale 114 is located adjacent the end point “WORST” of scale 112. The end point “BEST” of scale 114 is located adjacent the end point, “REMISSION” of scale 112. This scale 114 is the patient's subjective or visual analogue scale and in the drawing has been labelled VAS scale for easy reference by the patient.

The third scale 116 is on the opposite side of scale 112 from scale 114. The third scale 116 is entitled “JTCT” standing for active joint count. This scale has one end point “0” and one end point “28” . Aligned along the scale are the numbers 5, 10, 18. The end point, “0” of scale 116 is adjacent to the end point “REMISSION” of scale 112. The end point “28” is adjacent to the end point “WORST” of scale 112. Each of scales 114 and 116 are substantially parallel to scale 112 and the end points of the scales are aligned so that a line joining the respective end points is substantially perpendicular to the axis of 118 of scale 112.

In order to use the device of FIG. 1, the patient is first asked to mark a point on scale 114 which indicates the patient's self assessment of their present condition.

The physician or health care professional then does a proper study of the number of active joints of the patient which are affected by arthritis. In doing this test, the health care professional carries out an analysis of the patient's joints in accordance with accepted medical analysis. At the completion of the evaluation the health care professional then marks on the scale 116 the number of joints affected by the arthritic condition. By way of example, in FIG. 1, a health care professional has marked on scale 116, the point 130. Again, by way of example, FIG. 1 shows 3 points, 132, 134 andl36, any one of which would be marked by the patient on scale 114. Point 132 might be marked by a patient who was not feeling good about the present condition. Point 134 might be marked by a patient feeling about as expected and point 136 may be marked by a patient feeling better than expected. In each case, the patient would place a single mark on scale 114 before the health care professional marks scale 116.

A straight line is then used to join the point marked by the patient on scale 114 with the point marked by the health care professional on scale 116. A plurality of lines 150, 152 and 154 are drawn on the diagram to illustrate the connection between point 132, 134 or 136 marked by a patient and the point 130 marked by the health care provider after doing the objective analysis. Line 150 connects point 132 marked by a patient with point 130 while line 152 connects the point 134 with point 150 and line 154 connects point 136 with point 130. Points 132, 134 and 136 are illustrative only illustrating the patient's view of the same condition which has been assessed by the health care provider at point 130.

Arthritis is normally explained to a patient as being a condition which causes permanent damage and which cannot be reversed. The top of scale 112 above the remission end point is shown to the patient to indicate that damage will occur as a result of the condition. The tool is useful to demonstrate to the patient that unless the disease is brought under control the patient will continue to move down the scale 112 to the worst end point where there is a disability.

In the case of line 152 which connects points 134 and 130 together, it will be observed that this line is essentially perpendicular to the axis 118 of scale 112. This shows that the patient's appreciation for his condition is essentially accurate as determined by the clinical analysis performed by the health care professional. However, even in this case it is demonstrated to the patient that the patient is at the 50% level rather than at the remission end of the scale. The patient can then be counselled about alternate drug therapies or other treatments that may have clinical effectiveness and which would allow the patient to move up the ladder towards the remission end point and to decrease their active joint count as determined by clinical measure.

Line 154 illustrates a case in which a patient has rated his arthritic condition as close to very good while the clinical measure has determined 10 active joints. Lines 154 crosses axis 118 at an angle. In this case it is demonstrated to the patient that notwithstanding their own analysis of the situation, they are in fact lower down on the scale 112 as determined by the number of active joints. Typically patients in this situation will then undertake more aggressive therapy to treat their condition and to realize that they have to become more compliant with the therapy that has already been described in order to bring their disease under control.

Line 150 illustrates the situation when a patient has assessed his condition as being closer to the very poor end of the scale when in fact the active joint clinical analysis determined that the patient's condition has not deteriorated as much as the patient has assessed it. This illustrates what is the traditional case of when a patient states that they are worse off and the patient and health care provider normally are prepared to undergo a change from the current status. A change from the current status may involve more aggressive treatments to treat the disease or a better reliance on the treatment currently in place. Other reasons for the discrepancy (fibrositis, depression, etc.) also needs to be ruled out by the health care professional.

One of the reasons why the chart is particularly helpful in assessing and explaining to patients the nature of their state is that the tool is a simple and useful demonstration to the patient of where they may be between the ends of remission and worst, and to approach their treatment regimes as appropriate. This in turn encourages the patient to comply with the therapy prescribed by the health care professional to meet the goals attainable by that patient.

One of the benefits of this diagnostic tool is that the tool provides what is in effect a snap shot on the day the assessment is done. By providing this to the patient, patients are encouraged to improve compliance with the therapies currently being offered to them or to seek more aggressive treatment. As a series of assessments or snap shots are taken over time, there develops an album. The album effect provides graphical information showing the change in the patient's condition between successive visits. Over a period of time and with a number of snap shots taken it becomes much more apparent to the patient as to whether the goals being set are being met or whether the patient is staying the same or deteriorating. The series of snapshots illustrate clearly to the patient any trends. By way of example, if the slope of the connecting line remains the same, then the patient's subjective assessment has the same relationship to the clinical assessment. If the relationship is that demonstrated by line 152, the improvement or degeneration in the patient's condition will be readily apparent. If, however, on a first assessment a patient marks point 132 while the clerical assessor marks point 130 and on a second assessment, the patient marks point 136, while the clinical assessor again marks point 130, it can be easily demonstrated to the patient that despite the patient's subjective assessment, the arthritic condition is not moving toward remission. This will demonstrate to the patient that either different treatment is warranted or better adherence to the existing treatment is required.

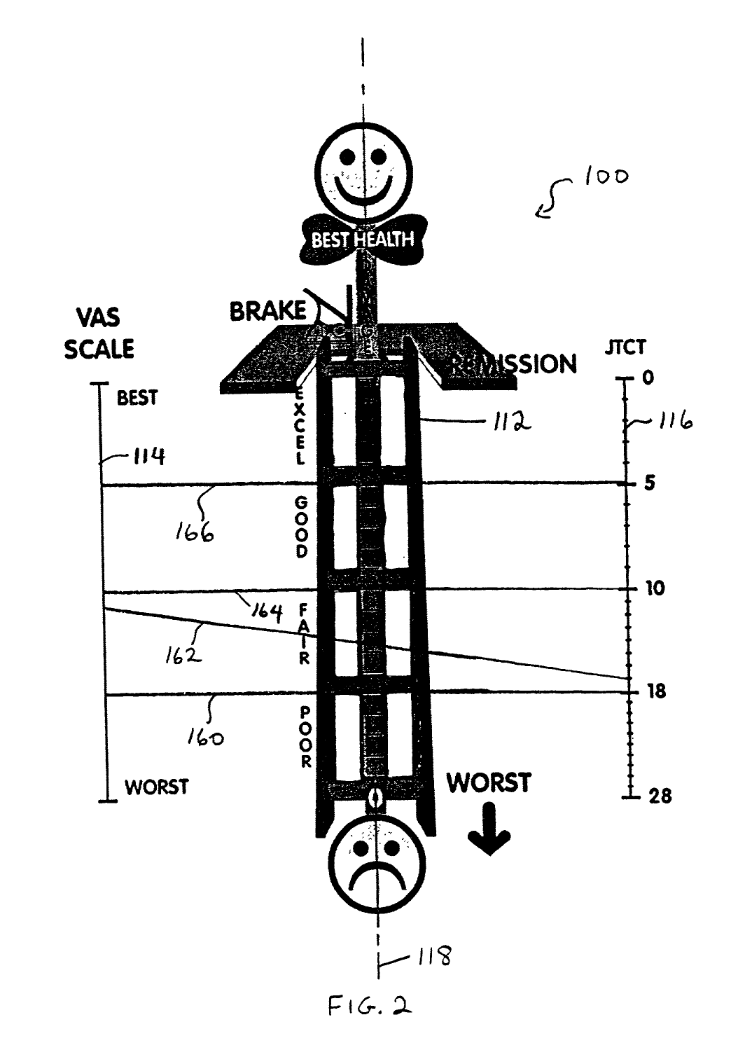

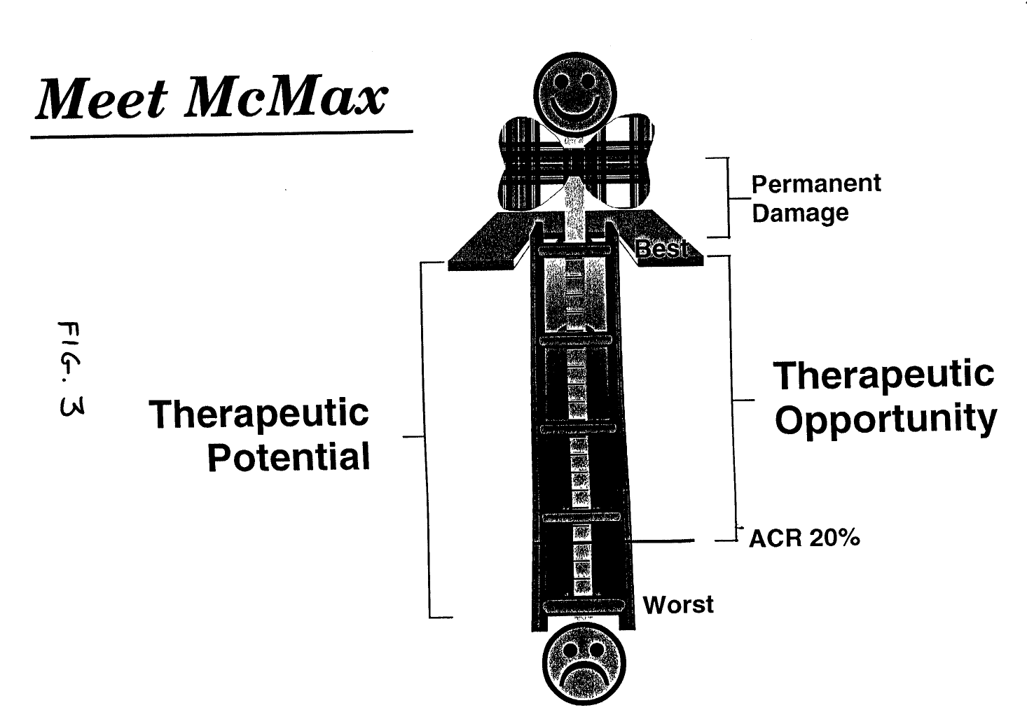

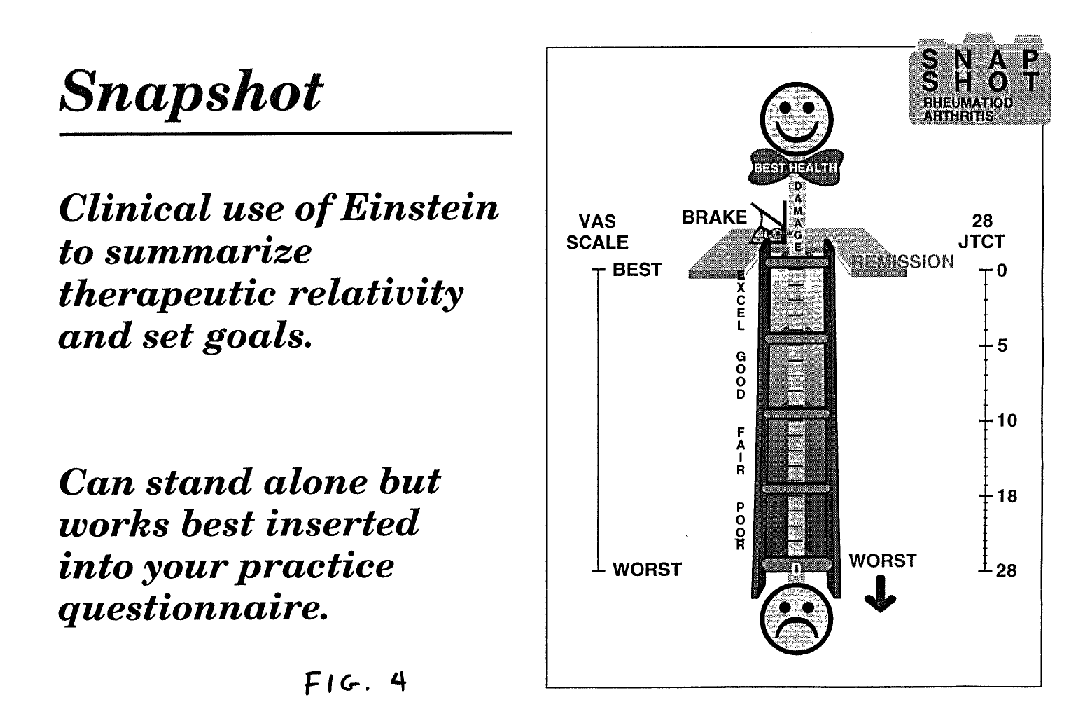

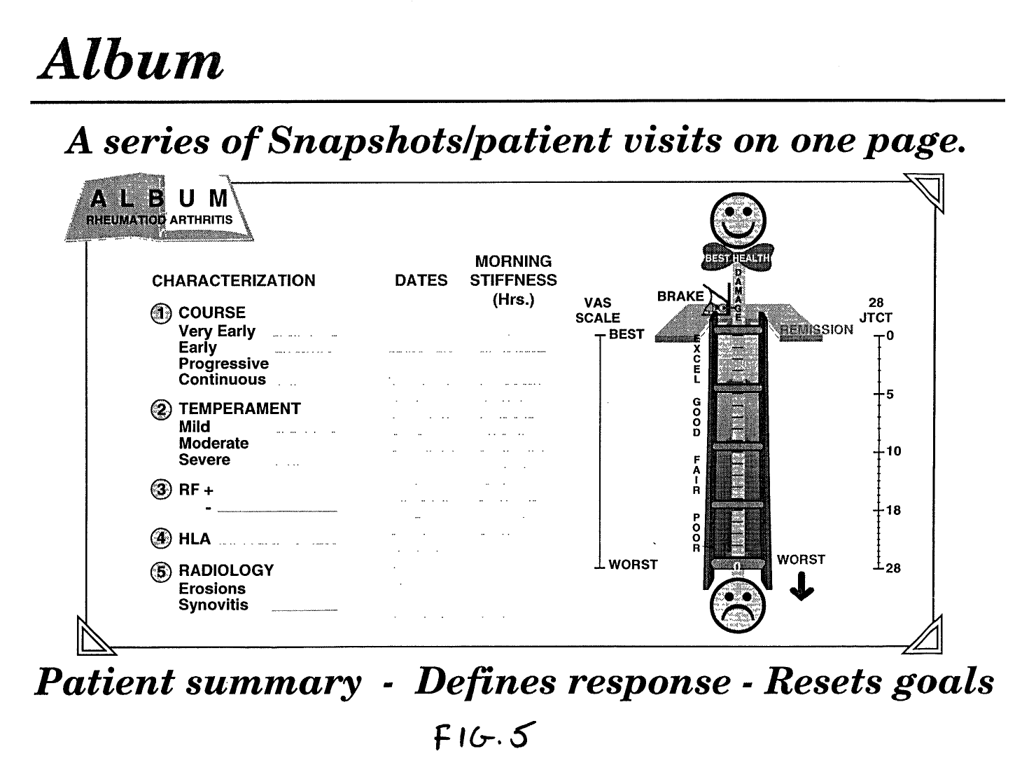

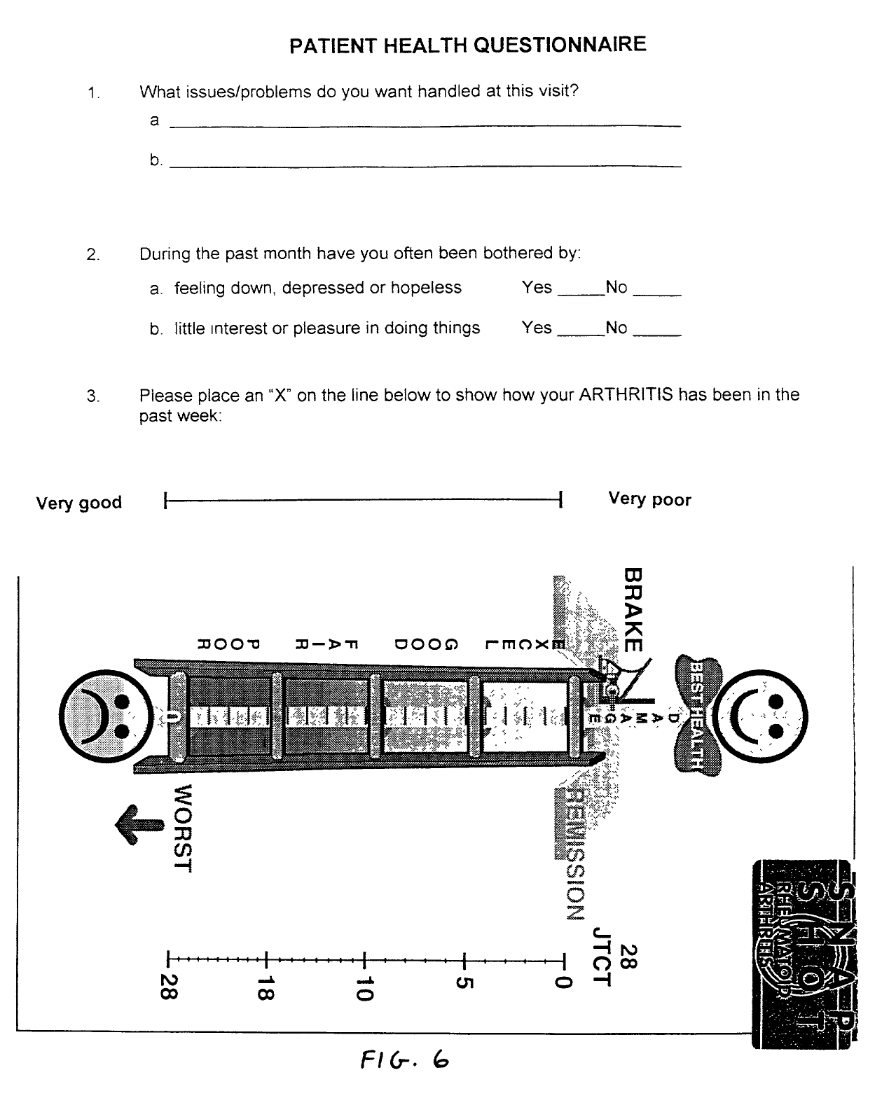

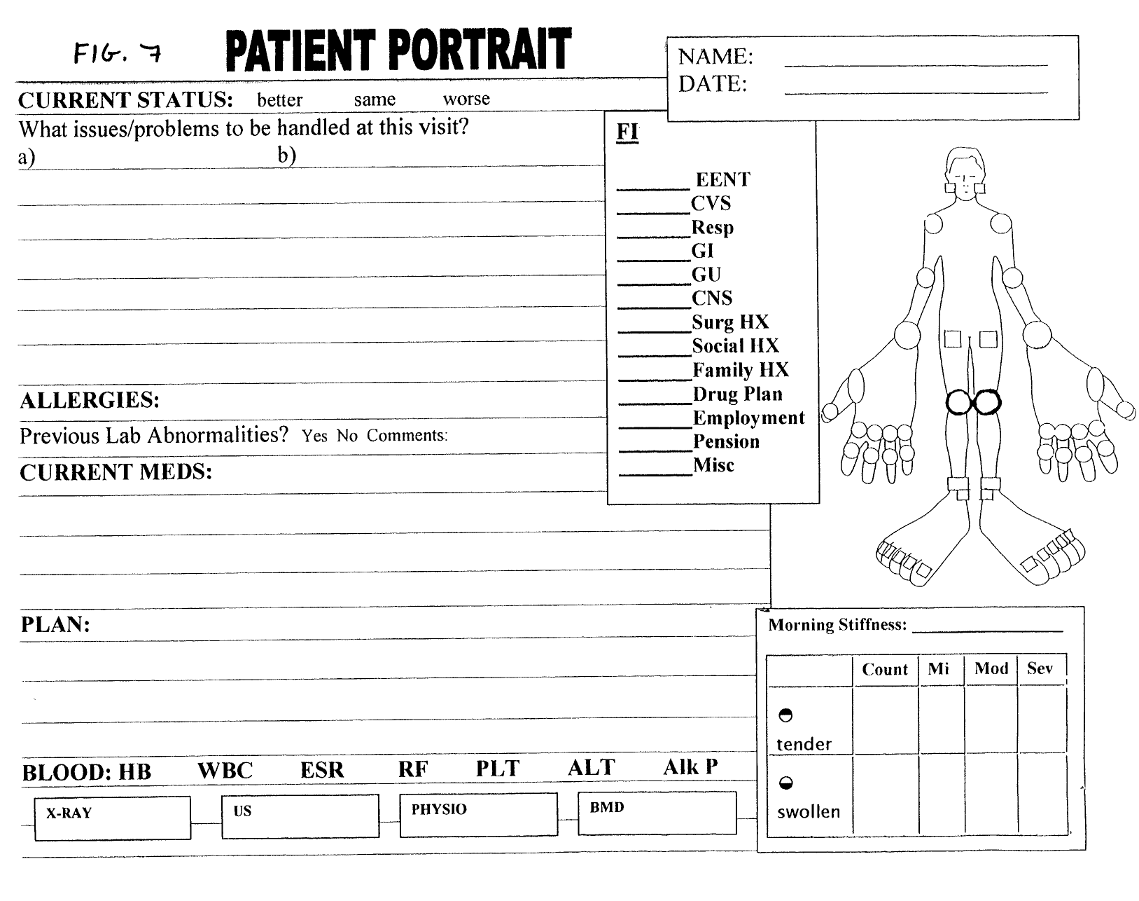

The album may be presented to the patient in the form of reproductions of the patient's snap shots taking during the course of treatment. Alternatively, to more clearly illustrate the progress or lack of progress, the lines generated during previous assessments or snapshots can be superimposed on a single drawing. This is shown in FIG. 2. FIG. 2 is a compilation of four snap shots. Line 160 represents the line drawn on a snap shot by the patient at a first assessment, line 162 represents a snap shot at the second patient assessment, line 164 represents a snap shot at the third patient assessment, and line 166 represents a snap shot of the patient at a fourth assessment.

Visits 1, 2, 3 and 4 may be spaced in accordance with the patient's regime. These assessments may be done yearly or, on a greater or lesser frequency as is appropriate for the patient and the patient's condition.

Line 160 is substantially perpendicular to the scales 112, 114 and 116 and shows the patient with a number of active joints and the patient himself is assessing his condition near the worst end of the scale. The active joint count, by way of example, is 18 for line 160.

Line 162 is created on the second assessment for this patient. At this time the patient has self assessed showing some improvement, whereas the joint assessment has shown only relatively minor improvement to a joint count of 17. By comparing lines 160 and 162 it is made clear to the patient that even though the patient feels there has been an improvement, in fact, the clinical assessment shows that there has been only been slight improvement.

Line 164 is substantially perpendicular to the scales 112, 114 and 116. The line 164 is generated by a patient who self-assessed at approximately the same level as the self-assessment during the second assessment. However, the clinical assessment shows that there has in fact been an improvement with the active joint count being reduced to 10.

Line 166, arising from the fourth assessment is also substantially perpendicular to scales 112, 114 and 116. This shows that the patient is feeling better and that the active joint count is also decreassing. Such an album encourages the patient to keep on with the treatment prescribed with a view to advancing to the remission end of the scale. This encouragement is vital in supporting the patient's own efforts so that the disease can be brought into the remission stage so that there will not be increased permanent damage.

In summary, the diagnostic tool is very useful in demonstrating to a patient, their current status and potential. A series of such charts also clearly demonstrates time trends so that the patient is motivated to move as close to remission as possible.

We claim:

1. A diagnostic tool comprising a plurality of scales,
said tool comprising a centrally located scale having a first axis,
said scale being marked by first and second end points,
said diagnostic tool comprising a second and third scale,
said second and third scales having axes generally parallel to said axis of said first scale,
said second and third scales being arranged on either side and substantially equidistant from said first scale,
said second scale having first and second end points,
said third scale having first and second end points,
said scales being arranged so that the line connecting said first end points is a straight line and is substantially perpendicular to said axis of said first scale and a line connecting said second end points poor is perpendicular to said axis of said first scale.

2. A diagnostic tool of claim 1 wherein said second scale is a visual analog scale.

3. The device of claim 1 in which said third scale is a clinical measure scale.

4. The device of claim 1 when the device is used to indicate a patient's medical condition.

5. The device of claim 4 wherein the medical condition is arthritis.
